(19) United States
(12) Patent Application Publication
(10) Pub. No.: US 2018/0056051 A1
(43) Pub. Date: Mar. 1, 2018

(54) APPARATUS AND METHODS FOR TREATING HARDENED VASCULAR LESIONS

(71) Applicant: Angioscore, Inc., Colorado Springs, CO (US)

(72) Inventor: Sameer Kabra, Fremont, CA (US)

(21) Appl. No.: 15/692,397

(22) Filed: Aug. 31, 2017

Related U.S. Application Data
(60) Provisional application No. 62/381,751, filed on Aug. 31, 2016.

(51) Int. Cl. A61M 29/02; A61B 17/3207

(52) U.S. Cl. CPC A61M 29/02; A61B 17/320725; A61B 2017/00778; A61B 17/320758; A61B 17/32075

(57) ABSTRACT

An angioplasty catheter comprises a catheter body having a balloon or other radially expandable shell at its distal end. A non-axial external structure is carried over the shell and scores a stenosed region in a blood vessel when the balloon is inflated therein. The catheter has an attachment structure disposed between the catheter body and the balloon to accommodate foreshortening and rotation of the external structure as the balloon is expanded. The external structure may be part of a helical cage structure which floats over the balloon.

### CROSS-REFERENCES TO RELATED APPLICATIONS

The present application claims the benefit of and priority to, under 35 U.S.C. § 119(e), co-pending U.S. Provisional Application Ser. No. 62/381,751, filed on Aug. 31, 2016, which is hereby incorporated herein by reference in its entirety for all that it teaches and for all purposes.

### BACKGROUND OF THE INVENTION

### 1. Field of the Invention

The present invention relates to the field of medical devices, more specifically to medical devices intended to treat stenoses in the vascular system.

Balloon dilatation (angioplasty) is a common medical procedure mainly directed at revascularization of stenotic vessels by inserting a catheter having a dilatation balloon through the vascular system. The balloon is inflated inside a stenosed region in a blood vessel in order to apply radial pressure to the inner wall of the vessel and widen the stenosed region to enable better blood flow.

In many cases, the balloon dilatation procedure is immediately followed by a stenting procedure where a stent is placed to maintain vessel patency following the angioplasty. Failure of the angioplasty balloon to properly widen the stenotic vessel, however, may result in improper positioning of the stent in the blood vessel. If a drug-eluting stent is used, its effectiveness may be impaired by such improper positioning and the resulting restenosis rate may be higher. This is a result of several factors, including the presence of gaps between the stent and the vessel wall, calcified areas that were not treated properly by the balloon, and others.

Conventional balloon angioplasty suffers from a number of other shortcomings as well. In some cases the balloon dilatation procedure causes damage to the blood vessel due to aggressive balloon inflation that may stretch the diseased vessel beyond its elastic limits. Such over inflation may damage the vessel wall and lead to restenosis of the section that was stretched by the balloon. In other cases, slippage of the balloon during the dilatation procedure may occur. This may result in injury to the vessel wall surrounding the treated lesion. One procedure in which slippage is likely to happen is during treatment of in-stent restenosis, which at present is difficult to treat by angioplasty balloons. Fibrotic lesions are also hard to treat with conventional balloons, and elastic recoil is usually observed after treatment of these lesions. Many long lesions have fibrotic sections that are difficult to treat using angioplasty balloons.

An additional problem associated with balloon angioplasty treatment has been the “watermelon seed effect.” Angioplasty is carried out at very high pressures, typically up to twenty atmospheres or higher, and the radially outward pressure of the balloon can cause axial displacement of the balloon in a manner similar to squeezing a watermelon seed with the fingers. Such axial displacement, of course, reduces the effectiveness of balloon dilatation. Another problem with conventional angioplasty balloon design has been deflation of the balloon. Even after the inflation medium is removed from a balloon, the deflated configuration will have a width greater than the original folded configuration which was introduced to the vasculature. Such an increase in profile can make removal of the balloon difficult.

Atherectomy/Thrombectomy devices intended to remove plaque/thrombus material may also include a structure that expands in a lesion while the plaque/thrombus removal mechanism is within this structure. The removed material is either being stacked in the catheter or sucked out thru the catheter. When the procedure is done, the expandable structure is collapsed and the catheter removed. Foreign object removal devices usually include a basket structure that needs to be expanded to collect the object and then collapse for retrieval. Distal protection devices usually include a basket structure that support a mesh that needs to be expanded distal to the treated lesion to collect the loose objects and then collapse for retrieval.

These devices usually include an elastic metallic material that needs to be expanded in the vascular system to fulfill its task and afterwards collapse to a small diameter to facilitate retrieval. The transition between the collapsed (closed) configuration to the expanded (open) configuration can be done in two ways: the structure can be at a normally closed (collapsed) configuration in which force is applied to cause the structure to expand. In this case, the elastic recoil of the structure will cause it to collapse back to closed configuration when the expanding force ceases. The structure may also be at a normally open (expanded) configuration in which a constraining element is forced over it to hold it down for the collapsed configuration (for example a constraining tube). When this constraining element is removed the structure is free to expand to the expanded (open) configuration. The structure material may also be non-lastic. In this case, the structure will need to be forced to transit between both collapsed and expanded configurations.

One problem associated with conventional angioplasty expansion systems is that the transition between the collapsed and expanded configurations involves significant rotational and axial reaction forces. These reaction forces are applied by the structure on the catheter as a result of the force applied by the catheter to expand or close the structure. Axial reaction forces are created due the foreshortening of the structure during expansion. Rotational reaction forces (torques) are created when a non-longitudinal element is forced to expand/collapse. Since the catheters are usually less stiff than the structure, these reaction forces may cause the structure to not expand or collapse properly, or cause undesired deformation to the catheter itself

To overcome at least some of these problems, U.S. Pat. No. 5,320,634 describes the addition of cutting blades to the balloon. The blades can cut the lesions as the balloon is inflated. U.S. Pat. No. 5,616,149 describes a similar method of attaching sharp cutting edges to the balloon. U.S. Patent Publication 2003/0032973 describes a stent-like structure having non-axial grips for securing an angioplasty balloon during inflation. U.S. Pat. No. 6,129,706 describes a balloon catheter having bumps on its outer surface. U.S. Pat. No. 6,394,995 describes a method of reducing the balloon profile to allow crossing of tight lesions. U.S. Patent Publication 2003/0153870 describes a balloon angioplasty catheter having a flexible elongate elements that create longitudinal channels in a lesion or stenosis.

While the use of angioplasty balloons having cutting blades has proved to be a significant advantage under many circumstances, the present cutting balloon designs and methods for their use continue to suffer from shortcomings. Most commercial cutting balloon designs, including those available from INTERVENTIONAL TECHNOLOGIES, INC., of San Diego, Calif., now owned by BOSTON SCIENTIFIC, of Natick, Mass., have relatively long, axially aligned blades carried on the outer surface of an angioplasty balloon. Typically, the blades are carried on a relatively rigid base directly attached to the outer balloon surface. The addition of such rigid, elongated blade structures makes the balloon itself quite stiff and limits the ability to introduce the balloon through torturous regions of the vasculature, particularly the smaller vessels within the coronary vasculature. Moreover, the cutting balloons can be difficult to deflate and collapse, making removal of the balloons from the vasculature more difficult than with corresponding angioplasty balloons which do not include cutting blades. Additionally, the axially oriented cuts imparted by such conventional cutting balloons do not always provide the improved dilatation and treatment of fibrotic lesions which would be desired.

For these reasons, it would be desirable to provide improved cutting balloon designs and methods for their use. In particular, it would be desirable to provide cutting balloons which are highly flexible over the length of the balloon structure, which readily permit deflation and facilitate removal from the vasculature, and which are effective in treating all forms of vascular stenoses, including but not limited to treatment of highly calcified plaque regions of diseased arteries, treatment of small vessels and/or vessel bifurcations that will not be stented, treatment of ostial lesions, and treatment of in-stent restenosis (ISR). Moreover, it would be desirable if such balloon structures and methods for their use could provide for improved anchoring of the balloon during dilatation of the stenosed region.

It would further be desirable to minimize the reaction forces applied by the external structure to the catheter, and at the same time be able to control the expansion of the expandable structure. It would also be desirable to adjust the compliance of the system in a predictable way without changing the materials or geometry of the expandable structure. At least some of these objectives will be met with the inventions described hereinafter.

### 2. Description of the Background Art

The following U.S. Patents and printed publication relate to cutting balloons and balloon structures: U.S. Pat. Nos. 6,450,988; 6,425,882; 6,394,995; 6,355,013; 6,245,040; 6,210,392; 6,190,356; 6,129,706; 6,123,718; 5,891,090; 5,797,935; 5,779,698; 5,735,816; 5,624,433; 5,616,149; 5,545,132; 5,470,314; 5,320,634; 5,221,261; 5,196,024; and Published U.S. Patent Application 2003/0032973. Other U.S. Patents of interest include U.S. Pat. Nos. 6,454,775; 5,100,423; 4,998,539; 4,969,458; and 4,921,984.

### SUMMARY OF THE INVENTION

The present invention provides improved apparatus and methods for the dilatation of stenosed regions in the vasculature. The stenosed regions will often include areas of fibrotic, calcified, or otherwise hardened plaque or other stenotic material of the type which can be difficult to dilatate using conventional angioplasty balloons. The methods and apparatus will often find their greatest use in treatment of the arterial vasculature, including but not limited to the coronary arterial vasculature, but may also find use in treatment of the venous and/or peripheral vasculature, treatment of small vessels and/or vessel bifurcations that will not be stented, treatment of ostial lesions, and treatment of ISR.

In a first aspect of the present invention, a scoring catheter comprises a catheter body having a proximal end and a distal end, a radially expandable shell (typically an angioplasty balloon) near the distal end of the catheter body, and a non-axial scoring structure carried over the shell. By “non-axial scoring structure,” it is meant that the structure will be able to score or cut stenotic material within a treated blood vessel along lines which are generally in a non-axial direction. For example, the scoring lines may be helical, serpentine, zig-zag, or may combine some axial components together with such non-axial components. Usually, the non-axial scoring pattern which is imparted will include scoring segments which, when taken in total, circumscribe at least a majority of and usually the entire inside wall of the blood vessel up to one time, preferably more than one time, usually more than two times, often at least three times, more often at least four, five, six, or more times. It is believed that the resulting scoring patterns which circumscribe the inner wall of the vessel will provide improved results during subsequent balloon dilatation.

Usually the scoring structure will comprise at least one continuous, i.e., non-broken, scoring element having a length of at least 0.5 cm, more usually at least 1 cm, often at least 2 cm, usually at least 3 cm, and sometimes at least 4 cm or more. Alternatively, the scoring structure may comprise a plurality of much smaller segments which may be arranged in a helical or other pattern over the balloon, typically having a length in the range from 0.1 cm to 2 cm, often being 0.5 cm or less, sometimes being 0.3 cm or less.

In order to promote scoring of the blood vessel wall when the underlying expandable shell is expanded, the scoring structure will usually have a vessel contact area which is 20% or less of the area of the expandable shell, usually being below 10%, and often being in the range from 1% to 5% of the area of the expandable shell. The use of a shell having such a relatively small contact area increases the amount of force applied to the vascular wall through the structure by expansion of the underlying expandable shell. The scoring structure can have a variety of particular configurations, often being in the form of a wire or slotted tube having a circular, square, or other cross-sectional geometry. Preferably, the components of the scoring structure will comprise a scoring edge, either in the form of a honed blade, a square shoulder, or the like. A presently preferred scoring edge is electropolished and relatively small.

In a preferred embodiment, the scoring structure may be formed as a separate expandable cage which is positioned over the expandable shell of the catheter. The cage will usually have a collar or other attachment structure at each end for placement on the catheter body on either side of the expandable shell. A collar may be a simple tube, and other attachment structures will usually be crimpable or otherwise mechanically attachable to the catheter body, such as a serpentine or other ring structure. The attachment structures on the cage may be attached at both ends to the catheter body, but will more usually be attached at only a single end with the other end being allowed to float freely. Such freedom allows the scoring structure to shorten as the structure is expanded on the expandable shell. In certain embodiments, both ends of the scoring structure will be fixed to the catheter body, but at least one of the attachment structures will have a spring or other compliant attachment component which provides an axial extension as the center of the scoring structure foreshortens.

In many cases, since the scoring elements are non-axial, there are torques induced during the expansion of the balloon and the shortening of the scoring structure. These torques may be high, and if one end of the scoring structure is constrained from rotation, the scoring element will not expand properly. The final expanded configuration of the scoring element is achieved via shortening and rotation.

In a preferred embodiment, both sides of the scoring element are fixed to the catheter, but at least one side will have a compliant structure which will provide axial tension and at the same time will allow the scoring element to rotate to its final configuration.

In some cases both ends of the scoring element are fixed and the shortening is achieved by deformation of the wire. For example, the wire can have a secondary structure which permits elongation (e.g., it may be a coiled filament) or can be formed from a material which permits elongation, e.g., nitinol. The scoring element can be attached in both ends, in a way that will allow rotation. In the case were the torques are low (depending on the design of the scoring element) there is no need for rotation and the torque can be absorbed either be the scoring element or by the catheter.

In all cases, the scoring structure is preferably composed of an elastic material, more preferably a super elastic material, such as nitinol. The scoring structure is thus elastically expanded over the expandable shell, typically an inflatable balloon similar to a conventional angioplasty balloon. Upon deflation, the scoring structure will elastically close to its original non-expanded configuration, thus helping to close and contain the balloon or other expandable shell.

In some cases the scoring element will be a combination of more than one material. In one case the scoring element can be made from nitinol and parts of it can be made from stainless steel. In other cases the scoring element can be made of stainless steel or nitinol and part of it can be made from polymer to allow high deformations.

In other preferred embodiments, the assembly of the shell and the scoring structure will be sufficiently flexible to permit passage through tortuous regions of the vasculature, e.g., being capable of bending at radius of 10 mm or below when advanced through 45 degrees, 90 degrees, or higher bends in the coronary vasculature. Usually, the scoring structure will comprise one or more scoring elements, wherein less than 70% of the cumulative length of the scoring element is aligned axially on the shell when expanded, preferably being less than 50% of the cumulative length, and more preferably being less than 25% of the cumulative length. In other instances, the scoring structure may comprise one or more scoring elements, wherein the cumulative length of the scoring element includes a non-axial component of at least 10 mm, preferably at least 12 mm, and more preferably 36 mm. Preferably, at least some of the scoring elements will have scoring edges which are oriented radially outwardly along at least a major portion of their lengths at all times during inflation and deflation and while inflated. By “radially outward,” it is meant that a sharp edge or shoulder of the element will be oriented to score or cut into the stenotic material or the interior wall of the treated vessel, particularly as the shell is being inflated.

The scoring elements will usually, but not necessarily, have a scoring edge formed over at least a portion of their lengths. A “scoring edge” may comprise a sharpened or honed region, like a knife blade, or a square shoulder as in scissors or other shearing elements. Alternatively, the scoring elements may be free from defined scoring edges, e.g., having circular or the other non-cutting profiles. Such circular scoring elements will concentrate the radially outward force of the balloon to cause scoring or other disruption of the plaque or other stenotic material being treated.

In a second aspect of the present invention, the scoring catheter comprises a catheter body and a radially expandable shell, generally as set forth above. The scoring structure will be composed of elements which circumscribe the radially expandable shell. By “circumscribing the radially expandable shell,” it is meant that at least some scoring elements of the scoring structure will form a continuous peripheral path about the exterior of the expandable shell during expansion. An example of such a fully circumscribing structure is a helical structure which completes up to one 360 degrees path about the balloon before, during, and after expansion, usually completing two complete revolutions, and frequently completing three, four, or more complete revolutions. Exemplary helical structures may include two, three, four, or more separate elements, each of which is helically arranged around the radially expandable shell.

In a third aspect of the present invention, a scoring catheter comprises a catheter body and a radially expandable shell, generally as set forth above. An elongated scoring structure is carried over the shell, and the assembly of the shell and the scoring structure will be highly flexible to facilitate introduction over a guide wire, preferably being sufficiently flexible when unexpanded so that it can be bent at an angle of at least 90 degrees, preferably 180 degrees, at a radius of 1 cm without kinking or otherwise being damaged. Such flexibility can be determined, for example, by providing a solid cylinder having a radius of 1 cm and conforming the assembly of the scoring structure and expandable shell over the cylinder. Alternatively, the assembly can be advanced over a guide wire or similar element having a 180 degrees one centimeter radius bend. In either case, if the assembly bends without kinking or other damage, it meets the requirement described above. Other specific features in this further embodiment of the catheters of the present invention are as described above in connection with the prior embodiments.

In a fourth aspect of the present invention, a plaque scoring catheter comprises a catheter body and a radially expandable balloon, generally as set forth above. A plurality of scoring elements are distributed over the balloon, typically being attached directly to an outer surface of the balloon. The scoring elements will be relatively short, typically having lengths below about 25% of the balloon length, preferably having lengths in the range from 2% to 10% of the balloon length. The relatively short, segmented scoring elements will permit highly flexible assemblies of balloon and scoring elements, generally meeting the flexibility requirement set forth above. The scoring elements may be arranged randomly over the balloon but will more usually be distributed uniformly over the balloon. In specific embodiments, the scoring elements may be arranged in helical, serpentine, or other regular patterns which circumscribe the balloon. As the balloon expands, such short segments will generally move apart from each other, but will still impart the desired scoring patterns into the vascular wall as the balloon is inflated.

In a fifth embodiment, the scoring catheter according to the present invention comprises a catheter body and a radially expandable balloon generally as set forth above. The balloon has a plurality of lobes extending between ends of the balloons, and at least one scoring element will be formed on at least one of the lobes in a manner arranged to score stenotic material as the balloon is expanded. The lobe will usually be in a helical pattern, and typically two, three, or more lobes will be provided. In the case of helical lobes, the scoring element(s) will usually be disposed along a helical peak defined by the helical lobe when the balloon is inflated. Such helical scoring elements will be arranged to accommodate balloon inflation, typically being stretchable, segmented, or the like.

In still another aspect of the apparatus of the present invention, an expandable scoring cage is adapted to be carried over a balloon of a balloon catheter. The scoring cage comprises an assembly of one or more elongate elastic scoring elements arranged in a non-axial pattern. As defined above, the non-axial pattern may comprise both axial and non-axial segments. The assembly is normally in a radially collapsed configuration and is expandable over a balloon to a radially expanded configuration. After the balloon is deflated, the assembly returns to a radially collapsed configuration, preferably being assisted by the elastic nature of the scoring cage. Advantageously, the scoring cage will enhance uniform expansion of the underlying balloon or other expandable shell and will inhibit “dog boning” where an angioplasty balloon tends to over inflate at each end, increasing the risk of vessel dissection. The scoring elements will be adapted to score hardened stenotic material, such as plaque or fibrotic material, when expanded by the balloon in a blood vessel lumen. The scoring cage may be adapted to mount over the balloon with either or both ends affixed to the balloon, generally as described above in connection with prior embodiments. Preferred geometries for the scoring elements include those which circumscribe the balloon, those which are arranged helically over the balloon, those which are arranged in a serpentine pattern over balloon and the like.

In yet another aspect of the present invention, a method for dilatating a stenosed region in a blood vessel comprises radially expanding a shell which carries a scoring structure. The scoring structure scores and dilates the stenosed region and includes one or more non-axial scoring elements arranged to impart a circumscribing score pattern about the inner wall of the blood vessel as the shell is expanded. The stenosed region is typically characterized by the presence of calcified plaque, fibrotic plaque, or other hardened stenotic material which is preferably scored prior to dilatation. Preferably, the scoring structure will not be moved in an axial direction while engaged against the stenosed region, and the scoring structure may optionally be free from axially scoring elements.

In still another aspect of the present invention, an angioplasty catheter comprises a catheter body and a radially expandable shell near the distal end of the catheter body. An external structure, such as a scoring structure or cutting structure, is carried over but unattached to the shell. The catheter further comprises an attachment structure having a proximal end and a distal end attached to the scoring structure, wherein the attachment structure is sufficiently sized and compliant to accommodate reaction forces or geometrical changes produced by the scoring structure as it is expanded by the shell. Generally, at least a portion of said scoring structure is arranged helically over the shell. However, the scoring structure may comprise numerous different configurations as described above.

In one aspect of the present invention, the proximal end of the attachment structure is fixed to the catheter body and the distal end of the attachment structure is secured to the proximal end of the scoring structure. In all cases, the attachment structure is capable axially and rotationally extending to accommodate foreshortening of the scoring structure as the shell is expanded.

In a preferred embodiment, the attachment structure comprises a compliance tube having an outer diameter and an inner diameter that extends over the catheter body. The inner diameter of the compliance tube is generally larger than an outer diameter of the catheter body so that the compliance tube freely extends and/or rotates with respect to the catheter body as the scoring structure foreshortens.

The compliance tube may also be sized to control the compliance of the scoring structure and expandable shell. Generally, the compliance tube has wall thickness ranging from 0.001 in to 0.1 in., preferably 0.005 in. to 0.05 in. The wall thickness may be increased to lessen the compliance of the system, or decreased to create a greater compliance. The length of the compliance tube may also be adjusted to control the compliance of the system. Generally, the compliance tube has a length ranging from 1 cm to 10 cm, but may range up to 30 cm or more for embodiments wherein the tube extends across the length of the catheter body.

In most cases, the material of the compliance tube may also be selected to control the compliance of the scoring structure and expandable shell. Generally, the compliance tube comprises an elastic material, preferably a polymer such as nylon or Pebax™ (available from Arkema Functional Polyolefins, Colombes Cedex, France). Alternatively, the compliance tube may comprise a braided material, metal or wire mesh.

In some aspects of the present invention, the compliance tube may have one or more perforations to control the compliance of the scoring structure and expandable shell. Generally, the perforations comprise one or more slots extending along the outside circumference of the compliance tube. The slots may form a pattern along the outside circumference of the compliance tube. The slots may be parallel to each other and/or extend helically or radially across the circumference of the compliance tube. The slots themselves may be formed of a variety of shapes, such as circular or rectangular.

Preferably, the compliance tube has an outer diameter that tapers from its distal end to its proximal end so that the outside diameter at the proximal end is slightly larger than the inner diameter, and the outside diameter at the distal end is sized to approximate the diameter of the scoring structure when in a collapsed configuration. This allows for the catheter to be readily removed from a vessel without catching or snagging on the vessel wall. For the tapered configuration, the outer diameter of the compliance tube will vary depending on the size of the catheter body and the expansion cage, but the diameter generally tapers down in the range of 0.004 in. to 0.010 in. from the distal end to the proximal end.

In another aspect of the invention, the attachment structure is connected at its distal end to the scoring structure and at its proximal end to a manipulator. Typically, the manipulator is positioned at the proximal end of the catheter body and the attachment structure extends from the scoring structure across the length of the catheter body. In all cases, the attachment structure is capable of axially and rotationally extending to accommodate foreshortening of the scoring structure as the shell is expanded.

In a preferred embodiment, the attachment structure comprises a compliance tube having an outer diameter and an inner diameter that extends over the catheter body. Typically, the inner diameter of the compliance tube is larger than an outer diameter of the catheter body so that the compliance tube freely extends and rotates with respect to the catheter body as the scoring structure foreshortens. The compliance of the scoring structure and expandable shell may be controlled by adjusting the thickness, length, or material selection of the compliance tube.

In some embodiments, the compliance of the scoring structure is controlled by actuating the manipulator during expansion or contraction of the radially expandable shell. Specifically, the attachment structure may be axially advanced with respect to the catheter body as the balloon is being inflated or deflated. For example, the attachment structure may be pulled away from the distal end of the catheter body while the balloon is being expanded to constrain the compliance of the balloon. Alternatively, the manipulator may be used to rotate the attachment structure with respect to the catheter body to control the compliance of the balloon during transition.

In another embodiment of the present invention, a method of dilatating a stenosed region in a blood vessel comprises introducing a scoring structure carried over an expandable shell that is connected to a catheter body by an attachment structure, and expanding the scoring structure within a stenosed region within the blood vessel. In this method, the attachment structure axially and/or rotationally extends to accommodate foreshortening of the scoring structure as the shell is expanded. The attachment structure generally comprises a compliance tube having an outer diameter and an inner diameter that extends over the catheter body, wherein the inner diameter of the compliance tube is larger than an outer diameter of the catheter body so that the compliance tube freely extends and rotates with respect to the catheter body as the scoring structure foreshortens. The thickness, length, and material of the compliance tube may be selected to control the compliance of the scoring structure and expandable shell.

In some embodiments, the method further comprises the step of fixing the proximal end of the attachment structure to the catheter body. Alternatively, the method may comprise the step of fixing the proximal end of the attachment structure to a manipulator. In such an embodiment, the manipulator is positioned at the proximal end of the catheter body and the attachment structure extends from the scoring structure across the length of the catheter body. This allows for the compliance of the scoring structure and balloon to be controlled by actuating the manipulator during expansion or contraction of the radially expandable shell. Actuation of the manipulator may occur by axially advancing, pulling, or rotating the attachment structure with respect to the catheter body.

### BRIEF DESCRIPTION OF THE DRAWINGS

FIGS. 1, 1A, 1B, and 1C are schematic illustrations of the balloon scoring structure embodiment in accordance with an embodiment of the invention.

FIG. 2 is a schematic illustration of an exemplary helical scoring structure embodiment in accordance with embodiments of the invention.

FIG. 3 is a schematic illustration of an expanded angioplasty balloon carrying a helical scoring structure in accordance with embodiments of the invention.

FIG. 4 illustrates a scoring structure comprising an alternating serpentine pattern of intermediate scoring elements between a pair of end collars.

FIG. 5 illustrates the serpentine scoring elements of the embodiment of FIG. 4 shown in a rolled-out configuration.

FIG. 6 illustrates a scoring structure comprising alternating C-shaped scoring elements between a pair of end collars.

FIG. 7 illustrates the C-shaped scoring elements of the embodiment of FIG. 6 shown in a rolled-out configuration.

FIG. 8 is a view of one of the C-shaped scoring elements taken along line 8-8 of FIG. 6.

FIG. 9 illustrates an alternative double C-shaped scoring element which could be utilized on a scoring structure similar to that illustrated in FIG. 6.

FIG. 10 illustrates an alternative embodiment of a helical scoring structure comprising serpentine and zigzag structures for mounting onto a balloon catheter.

FIG. 11 illustrates a first of the serpentine mounting elements of the scoring structure of FIG. 10.

FIG. 12 illustrates a second of the serpentine mounting elements of the scoring structure of FIG. 10

FIG. 13 illustrates an alternative mounting structure for a helical or other scoring structure.

FIG. 14 illustrates the mounting structure of FIG. 13 shown in a rolled-out configuration.

FIG. 15 shows yet another embodiment of a mounting element for the scoring structures of the present invention.

FIG. 16 illustrates the mounting structure of FIG. 15 shown in a rolled-out configuration.

FIG. 17a illustrates yet another alternative embodiment of a catheter constructed in accordance with the principles of the present invention, where an attachment structure is disposed between the scoring structure and the catheter body.

FIG. 17b illustrates the structure of FIG. 17a shown without the balloon.

FIG. 18 illustrates an embodiment of the invention having a laminated section at the distal end of the compliance tube.

FIG. 19 illustrates another view of the embodiment of FIG. 18.

FIG. 20 illustrates the embodiment of FIG. 18 with an expandable balloon inserted within the scoring structure.

FIG. 21 illustrates an embodiment with a sleeve over the distal end of the scoring structure.

FIG. 22 illustrates a method of the present invention utilizing an insertion tube to mount the scoring structure over the expandable balloon.

FIG. 23 illustrates shows the insertion tube inserted over the expandable balloon.

FIG. 24 illustrates a scoring catheter of the present invention with the insertion tube removed.

FIG. 25 illustrates yet another alternative embodiment of an angioplasty catheter constructed in accordance with the principles of the present invention.

FIG. 25A is a detail view of the catheter within line 25A-25A of FIG. 25; a dilatation balloon and an external structure of the catheter are illustrated in a collapsed configuration.

FIG. 25A′ is a detail view of the catheter within line 25A-25A of FIG. 25; the dilatation balloon and the external structure of the catheter are illustrated in an expanded configuration, and an attachment structure of the catheter is removed.

FIG. 26 illustrates the catheter of FIG. 25 with the external structure and the attachment structure removed and the dilatation balloon in the expanded configuration.

FIG. 26A is a detail view of the catheter with line 26A-26A of FIG. 26.

FIG. 26B is a detail view of a rapid exchange port of the catheter with line 26B-26B of FIG. 26A.

FIG. 27A is a side view of the attachment structure of the catheter of FIG. 25.

FIG. 27B is an end view of the attachment structure of FIG. 27A.

FIG. 28 illustrates yet another alternative embodiment of an angioplasty catheter constructed in accordance with the principles of the present invention.

FIG. 28A is a detail view of the catheter within line 28A-28A of FIG. 28; an expandable shell and an external structure of the catheter are illustrated in a collapsed configuration.

FIG. 28A′ is a detail view of the catheter within line 28A-28A of FIG. 28; the expandable shell and the external structure of the catheter are illustrated in an expanded configuration.

FIG. 28B is a detail view of the catheter within line 28B-28B of FIG. 28.

FIG. 29 is another view of the angioplasty catheter of FIG. 28; an expandable shell of the catheter is removed.

FIG. 29A is a detail view of the catheter within line 29A-29A of FIG. 29; the expandable shell and the external structure are illustrated in the collapsed configuration.

FIG. 29B is a detail view of the catheter within line 29B-29B of FIG. 29.

FIG. 29C is a detail view of the catheter within line 29C-29C of FIG. 29A.

FIG. 30 illustrates the catheter of FIG. 28 with the expandable shell in the expanded configuration, and the external structure and an attachment structure of the catheter removed.

FIG. 30A is a detail view of the catheter with line 30A-30A of FIG. 30.

FIG. 30B is a detail view of a rapid exchange port of the catheter with line 30B-30B of FIG. 28.

FIG. 30C is a cross section view of an inner sheath of the catheter along line 30C-30C of FIG. 30B.

FIG. 31A is a side view of the attachment structure of the catheter of FIG. 28.

FIG. 31B is an end view of the attachment structure of FIG. 31A.

FIG. 32 is a side view of a proximal sheath of the catheter of FIG. 28.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Embodiments of the present invention relate to device for revascularization of stenotic vessels and specifically to a balloon catheter having external elements. The dilatation device comprises a conventional dilatation balloon such as a polymeric balloon and a spiral, or external elements with other configurations mounted on the balloon catheter.

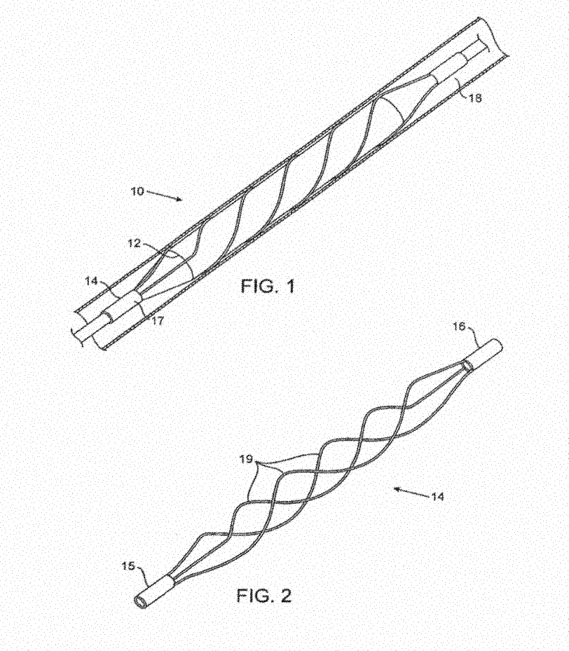

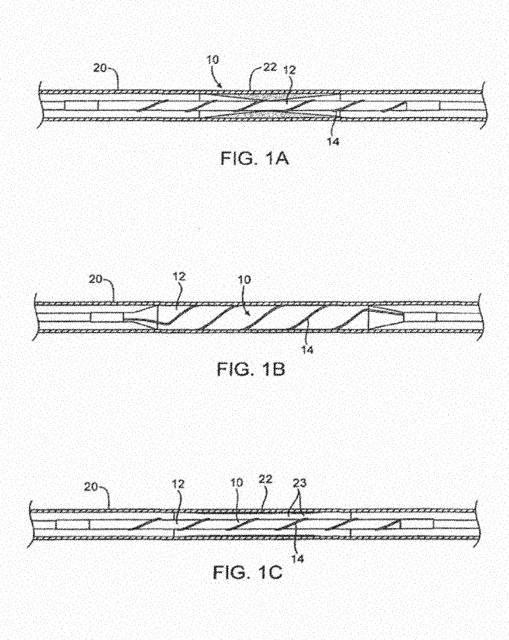

Reference is now made to FIGS. 1, 1A, and 1B, which are schematic illustrations of a dilatation device 10 in accordance with embodiments of the invention. The dilatation device 10 includes a dilatation balloon 12, which may be any conventional angioplasty balloon such as commonly used by interventional cardiologists or radiologists, and a helical or spiral unit 14 mounted over or attached to dilatation balloon 12. The compliance of the balloon and the scoring element(s) should be chosen to assure uniform expansion of the balloon substantially free from “dog-boning” as the combined structure expands within a lesion. If a compliant or a semi-compliant balloon is used and the compliance of the scoring element was not matched to comply with the properties of the balloon, the expansion of the balloon-scoring element system will not be uniform. This non-uniformity may impair the efficacy of the scoring catheter and, in some cases, may result in poor performance. For example, under given pressure, certain parts of the balloon will be able to expand while other parts will be constrained by excessive resistance of the scoring elements.

Helical unit 14 is typically made of nitinol. Helical unit 14 may be made of other metals such stainless steel, cobalt-chromium alloy, titanium, and the like. Alternatively, spiral unit 14 may be a polymeric spiral, or made of another elastic material. Helical unit 14 may be attached at its proximal and distal ends to the proximal end 17 and distal end 18 of dilatation balloon 12. Alternatively, spiral unit 14 may be attached to the distal end and/or the proximal end of dilatation balloon 12 by collar-like attachment elements 15 and 16. Spring or other compliant elements may be alternatively or additionally provided as part of the attachment elements to accommodate shortening of the helical unit as it is expanded.

Dilatation device 10 is inserted into the vascular system, for example, using a conventional catheter procedure, to a region of stenotic material 22 of blood vessel 20. (The term “stenotic” is used herein to refer to the vascular lesion, e.g., the narrowed portion of the vessel that the balloon is meant to open.) At the stenotic area, the dilatation balloon 12 is inflated, for example, by liquid flow into the balloon. Helical unit 14 widens on the inflated dilatation balloon 12. On inflation, the dilatation balloon 12 together with the helical unit 14 is pressed against the walls of blood vessel 20 as shown in FIG. 1B.

Reference is now made to FIG. 1C, illustrating blood vessel 20 after the deflation of dilatation balloon 12. Helical unit 14 narrows when deflating the dilatation balloon 12, thus the dilatation device 10 is narrowed and may be readily retrieved from blood vessel 20. The deflation profile of the balloon 10 is low and mainly circular. The stenotic material 22 in blood vessel 20 is pressed against blood vessel 20 walls to widen the available lumen and enhance blood flow. The pressing of helical unit 14 against the walls of blood vessel 20 causes scoring 23 in the stenotic area

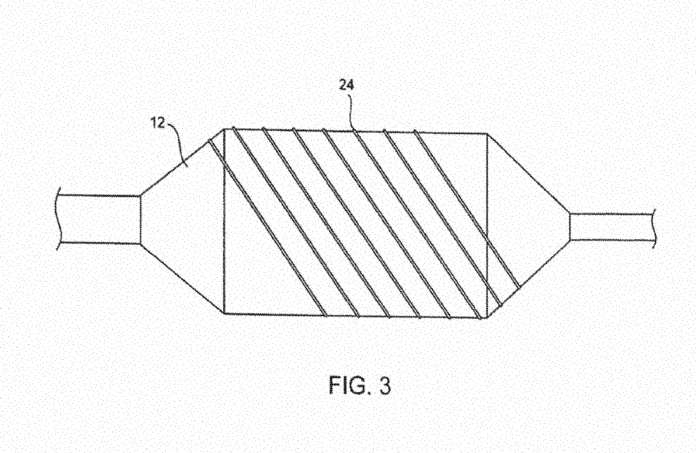

Reference is now made to FIG. 3 that shows a scoring structure in the form of a single wire 24 wrapped around a dilatation balloon 12 in a helical configuration.

In other embodiments, the scoring structure of the present invention can have a non-helical configuration. Any design of scoring structure that can accommodate an increase in the diameter of the balloon 12 upon inflation, and return to its configuration when the balloon is deflated, is an appropriate design useful in the invention. At least a portion of the scoring elements will not be parallel to the longitudinal axis of the balloon catheter to enhance flexibility and improve scoring.

Referring again to FIGS. 1A-1C, helical unit 14 is pushed outwardly by the inflation of the balloon 12, and is stretched by the inflation of the balloon. When the balloon is deflated, helical unit 14 assists in the deflation by its elastic recoil. This active deflation is faster and also leads to a low profile of the deflated balloon. The balloon 12 is disposed within the helical unit 14, which returns to its pre-inflated shape and forces the balloon to gain a low radial profile.

In another embodiment of the invention, dilatation device 10 may carry a stent. The stent can be crimped over the helical unit 14. In this way, the helical unit 14 can push the stent against hard areas of the lesion, enabling proper positioning of the stent against the vessel wall, even in hard-calcified lesions without pre-dilation.

Reference is now made to FIG. 2, illustrating the helical unit 14 in accordance with embodiments of the invention. Helical unit 14 is typically made of nitinol. Helical unit 14 includes three wires 19 that are attached to collars 15 and 16 at the proximal end and distal end, respectively. Alternatively the scoring structure may be formed as a metallic cage, which can be made from a slotted tube, or polymeric cage or polymeric external elements. Alternatively, the scoring structure may comprise wires of other elements attached directly to the balloon material or close to the balloon ends.

Wires 19 (FIG. 2) are attached between collars 15 and 16. The diameter of the wires is typically in the range of 0.05 mm to 0.5 mm. Alternatively, a cage (for example a metallic cage made of a slotted tube) can be used in several configurations that allow local stress concentrations. The size and shape of the cross section of the cage elements or the cross section of the wires can vary. The cross section can be a circle, rectangle, triangle, or other shape.

In alternative embodiments, the wires 19 may comprise short segments that are attached to the balloon 12.

In further alternative embodiments of the invention, the helical unit 14 may be glued, thermally bonded, fused, or mechanically attached at one or both ends to dilatation balloon 12.

In yet another embodiment, a scoring structure may comprise wires that are attached to the dilatation balloon 12 in a helical configuration or other configuration. The wires may be thermally attached to the balloon 12, glued, mechanically attached, or the like.

In still another embodiment, a scoring structure comprises wire or cage elements that are not parallel to the longitudinal axis of the balloon 12 so that the combination of the scoring structure 19 and the balloon 12 remains flexible.

In additional embodiments, the combination of dilatation balloon 12 and scoring structure scores the lesion and provides better vessel preparation for drug eluting stents by allowing better positioning of the stent against the vessel wall and diffusion of the drug through the scores in the lesion.

In these embodiments, the balloon can be used as a platform to carry drugs to the lesion where scoring of the lesion can enhance delivery of the drug to the vessel wall.

In these embodiments, the balloon can be used for a local drug delivery by embedding drug capsules, drug containing polymer, and the like, through the stenotic material and into the vessel wall.

From the above, it can be seen that the invention comprises catheters and scoring structures, where the scoring structures are positioned over the balloons or other expandable shells of the catheter. The scoring structures may be attached directly to the balloons or other shells, in some cases being embedded in the balloon material, but will more usually be formed as separate cage structures which are positioned over the balloon and attached to the catheter through attachment elements on either side of the balloon. The expandable cages may be formed using conventional medical device fabrication techniques, such as those used for fabricating stents, such as laser cutting of hypotube and other tubular structures, EDM forming of hypotubes and tubes, welding of wires and other components and the like.

Typically, such expandable shell structures will comprise the attachment elements and an intermediate scoring section between the attachment elements. As illustrated in the embodiments above, the attachment elements may be simple cylindrical or tube structures which circumscribe the catheter body on either side of the balloon or other expandable shell. The simple tube structures may float over the catheter body, i.e., be unattached, or may be fixed to the catheter body. A number of alternative embodiments for the attachment elements will be described in connection with the embodiments below.

The intermediate scoring sections may also have a variety of configurations where at least some of the scoring elements will typically be disposed in a non-axial configuration, i.e., in a direction which is not parallel to the axial direction of the expandable cage. A preferred configuration for the intermediate scoring section comprises one or more helical elements, generally as illustrated in the prior embodiments. Other exemplary configurations are set forth in the embodiments described below.

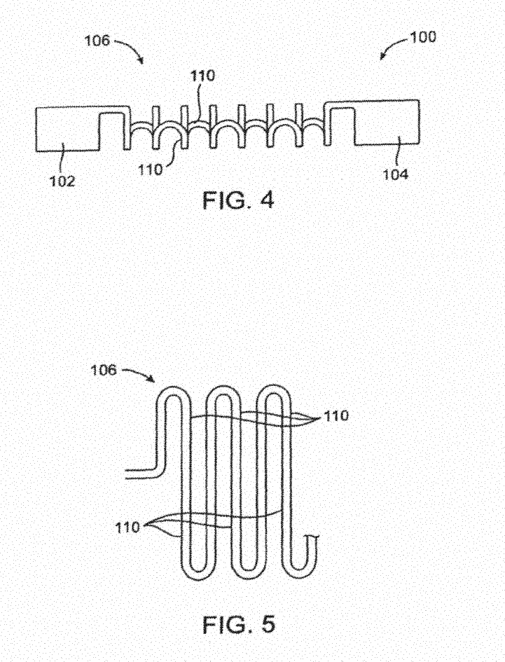

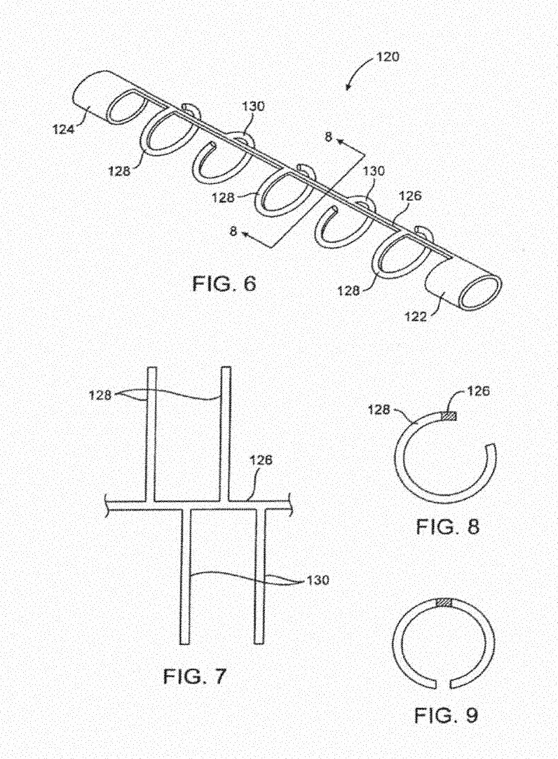

Referring now in particular to FIGS. 4 and 5, an expandable scoring cage 100 comprises first and second attachment elements 102 and 104, respectively, and an intermediate scoring section 106 comprising a plurality of curved serpentine members 110. The serpentine members 110 extend circumferentially in opposite directions in an alternating manner. This can be understood by observing a “rolled-out” view of the serpentine elements as illustrated in FIG. 5. A second alternative scoring cage structure 120 is illustrated in FIGS. 6-8. The scoring cage 120 comprises first and second attachment elements 122 and 124 joined by a spine 126. A plurality of C-shaped scoring elements 128 and 130 are attached to the spine and extend in opposite circumferential directions. The shape of the element can be observed in FIG. 8. The opposite directions may be observed in the rolled-out view of FIG. 7.

It will be appreciated that a variety of different circumferential structures may be used in place of the C-shaped structures of FIGS. 6-8. For example, a pair of opposed C-shaped partial ring structures may be utilized, as illustrated in FIG. 9. The C-shaped structures of FIG. 6 or the double C-shaped structures of FIG. 9 can also be extended so that they wrap around a balloon more than one time, either over or under the spine structure 126.

The expandable cage structures 100 and 120 will each be mounted over a dilatation balloon, such as the balloon of FIGS. 1-3, with the attachment elements secured to the catheter body on either side of the dilatation balloon. The tube or cylindrical attachment elements 102, 104, 122, and 124 may simply float over the catheter body. In other embodiments, however, it may be desirable to use an adhesive or other means for affixing either one or both of the attachment elements to the catheter body. Having at least one floating attachment element, however, is often desirable since it can accommodate shortening of the intermediate scoring section as that section radially expands. In other cases, however, the individual scoring elements may possess sufficient elasticity to accommodate such shortening. For example, nitinol and other shape memory alloys possess significant stretchability, typically on the order of 8%, which in some instances will be sufficient to accommodate any tension applied on the intermediate scoring section by radial expansion of the balloon.

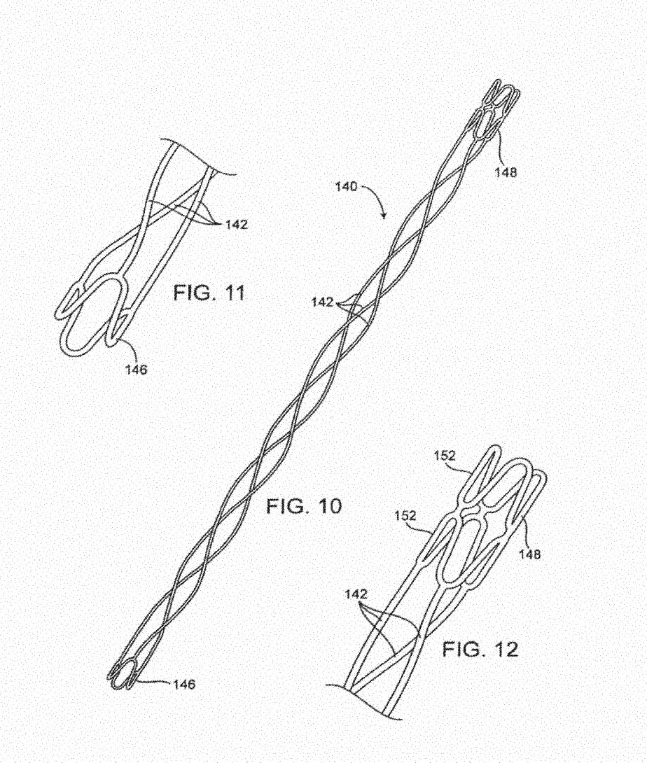

Referring now to FIGS. 10-12, alternative attachment elements are shown on an embodiment of an expandable scoring cage 140 comprising three helical scoring elements 142 which make up the intermediate scoring section. A first attachment element 146 comprises a single serpentine ring, as best illustrated in FIG. 11 while a second attachment element 148 comprises a pair of tandem serpentine rings 150 and 152, as best illustrated in FIG. 12. The use of such serpentine attachment structures is beneficial since it permits crimping of either or both of the structures onto the catheter body in order to fix either or both ends of the structure thereto. Usually, the single serpentine attachment structure 146 will be affixed to the catheter body while the double serpentine structure will be left free to allow movement of that end of the scoring cage to accommodate radial expansion of the underlying balloon.

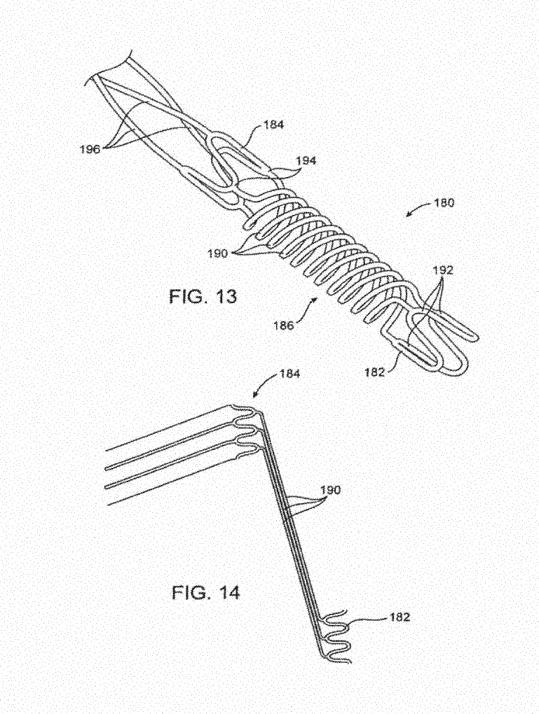

Referring now to FIGS. 13 and 14, a further alternative embodiment of an attachment element useful in the scoring cages of the present invention is illustrated. Attachment element 180 includes a pair of serpentine rings 182 and 184, generally as shown in FIG. 13, in combination with a coil spring structure 186 located between said rings 182 and 184. The coil spring structure 186 includes three nested coil springs 190, each joining one of the bend structures 192 and 194 on the serpentine rings 182 and 184, respectively. The structure of the spring structure and adjacent serpentine rings can be understood with reference to the rolled-out configuration shown in FIG. 14.

The attachment structure 180 is advantageous since it permits a fixed attachment of the outermost ring 182 to the underlying catheter body while the inner ring 184 remains floating and expansion and contraction of the intermediate scoring section, comprising helical elements 196, is accommodated by the coil spring structure 186. Since the scoring cage is fixed to the catheter, any risk of loss or slippage from the balloon is reduced while sufficient compliance is provided to easily accommodate radial expansion of the intermediate scoring section. By attaching the structures 180 at at least one, and preferably both ends of the scoring cage, the risk of interference with a stent is reduced.

In some embodiments, collars, such as those shown in FIGS. 1 and 2, or attachment elements, such as those shown in FIGS. 10-12, may comprise a flexible material that allows the collar or attachment element to expand while being mounted over the balloon catheter and then be collapsed to the diameter of the catheter. The expandability of the collars and/or attachment elements may be achieved by a compliant memory material such as nitinol or a polymer, or by use of a flexible serpentine design as shown in FIGS. 10-12. Where collars are used, the collar may be shaped or have a slit down the circumference (not shown) so that the collar may be expanded during mounting over the balloon. Alternatively, the collar may be oversized to accommodate the balloon diameter mounting, and then crimped down to secure the secure the scoring structure to the catheter body.

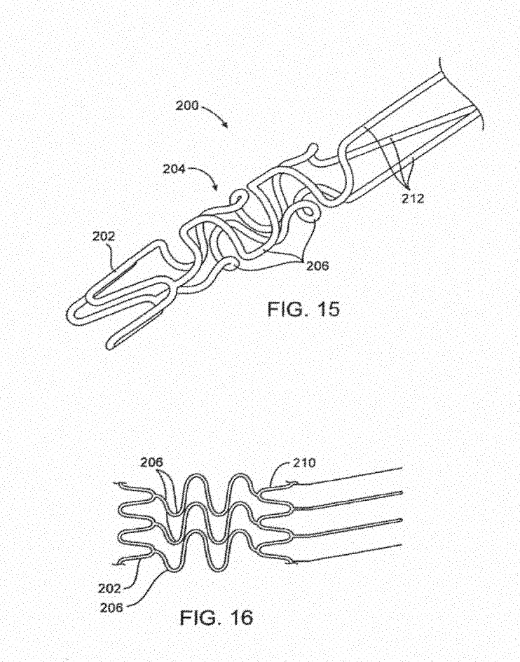

Yet another embodiment of the attachment element of the present invention includes an axial spring as shown in FIGS. 15 and 16. The attachment element 200 includes a terminal serpentine ring 202 and an intermediate spring structure 204 including a number of axial serpentine spring elements 206. The nature of the serpentine ring elements 206 can be observed in the rolled-out configuration of FIG. 16. Optionally, a second serpentine ring 210 may be provided between the attachment structure 200 and the helical scoring elements of the intermediate scoring section 212.

The embodiments of FIGS. 13-16 comprise spring-like elements 186 and 204 to accommodate axial shortening of the scoring structure upon radial expansion. It will be appreciated that other metal and non-metal axially extensible structures could also be used in such attachment structures. For example, elastic polymeric tubes could be attached at one end to the scoring structures and at another end to the catheter body (or to a ring, collar or other structure which in turn is fixed to the catheter body).

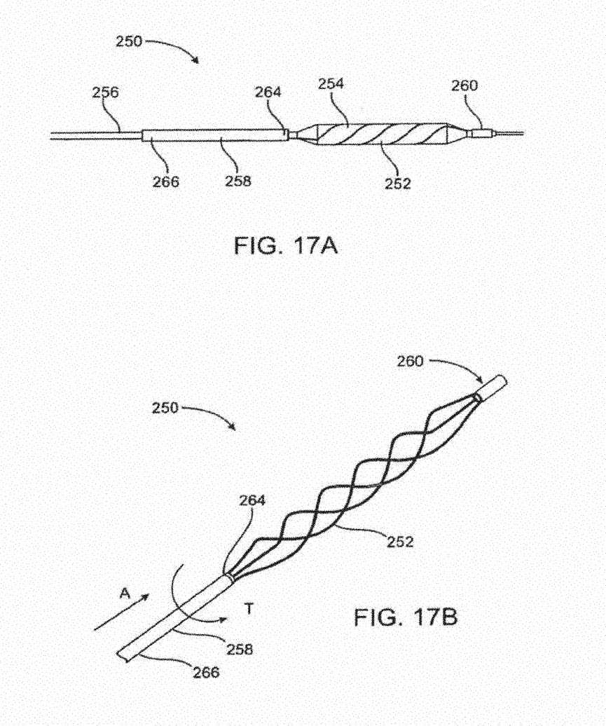

Referring now to FIGS. 17a and 17b, a further embodiment of an angioplasty catheter 250 having an axially distensible attachment structure 258 is illustrated. External structure 252 is held over expandable dilatation balloon 254 and is fixed at one end to the distal end 260 of catheter body 256. The external structure may comprise any structure typically used for removal of plaque/thrombus from a vessel wall such as a scoring structure, cutting structure, or crushing structure. The proximal end 262 of external structure 252 is connected to the distal end 264 of attachment structure 258. The proximal end 266 of attachment structure 258 is fixed to the catheter body 256. As described below, the attachment structure 258 may be configured to reduce forces applied on the external structure 252 and the catheter body 256 during expansion and contraction of balloon 254.

In a preferred embodiment, attachment structure 258 comprises a cylindrical over-tube, or compliance tube, made of an elastic material. Over-tube 258 generally has an inner diameter that is slightly greater than the outer diameter of the catheter body 256. Because only a small section of the proximal end of the attachment structure 258 is fixed to the catheter body, the distal end 264 attached to external structure 252 is free floating, and is free to slide axially and rotationally with respect to catheter body 256. Attachment structure 252 may be fixed, for example by adhesion, directly to the catheter body 256 and external structure 252, or to a collar or other intermediate attachment means.

As balloon 254 is expanded, external structure 252 expands in circumference and contracts axially along the catheter body 256, creating axial force A on attachment structure 258. Attachment structure 258, fixed to the catheter at its end 266, axially stretches to accommodate the axial movement of the external structure 252. External structure 252 also tends to rotate about the catheter body 256, causing a torsional force T. The distal end 264 of attachment structure 258 rotates through the full range of motion of scoring structure 252 to accommodate torsional force T, while proximal end 266 remains stationary with respect to catheter body 256.

The configuration illustrated in FIGS. 17a and 17b allows the compliance of the expandable system to be controlled. Generally, where one end of the scoring structure is free, the compliance of the expandable system will be a combination of the compliance of the balloon and the scoring structure. However, because the ends of the expandable system shown in FIG. 17 are fixed at distal end 260 and proximal end 266, the attachment structure controls the compliance of the expandable system.

The compliance of the system may be varied by any combination of material selection, wall thickness, or length of the over-tube 258. Over-tube 258 may comprise any elastomer, such as elastic polymer like Nylon, Pebax™, or PET. Typically, compliance tube 258 is formed from extruded tubing, but it may also comprise braided polymeric or metallic fibers, or wire mesh. A high memory metal such as nitinol or stainless steel may also be used. Where the compliance tube comprises an extruded polymeric tube, the wall thickness can vary in the ranges set forth above, and the length of the tube can range from 1 cm to 10 cm. For the same material, the thinner-walled and longer the tube, the more compliant the system.

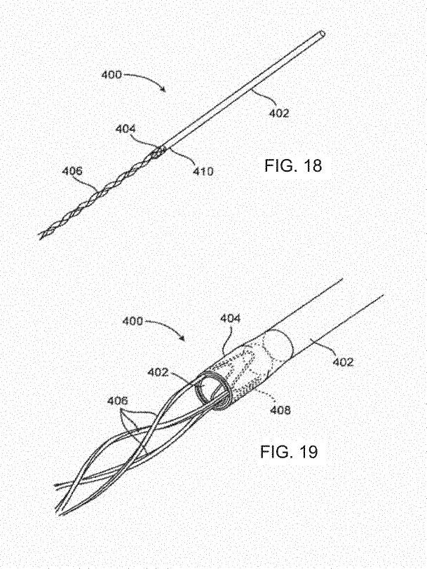

Now referring to FIGS. 18 and 19, a scoring cage structure 400 is illustrated having a two-layer laminated compliance tube 402. As shown in FIG. 19, the compliance tube 402 has a laminated structure 404 at at least its distal end 410. The laminated structure holds the proximal ends 408 of the scoring elements 406 as shown in broken line in FIG. 19. The scoring elements 406 may be sized to fit over the outside of the compliance tube 402, as illustrated in FIG. 19, with the lamination covering the elements. Alternatively, the compliance sleeve tube 402 may be sized to fit inside of the scoring structure 406, with the laminating layer(s) formed over the elements 406 (not shown).

The laminating structure may be composed of a polymer similar to the compliance tube 402, and may be heat shrunk or melted to thermally bond the compliance sleeve to the compliance tube and sandwich the scoring structure 406. Alternatively, an adhesive or other bonding method such as ultrasonic or RF energy may be used to laminate the structure. The laminated structure, as shown in FIGS. 18 and 19, provides a smoothed transition and strengthened bond between the scoring cage and the attachment structure. Such a smooth transition is a particular advantage when withdrawing the scoring cage from the vasculature.

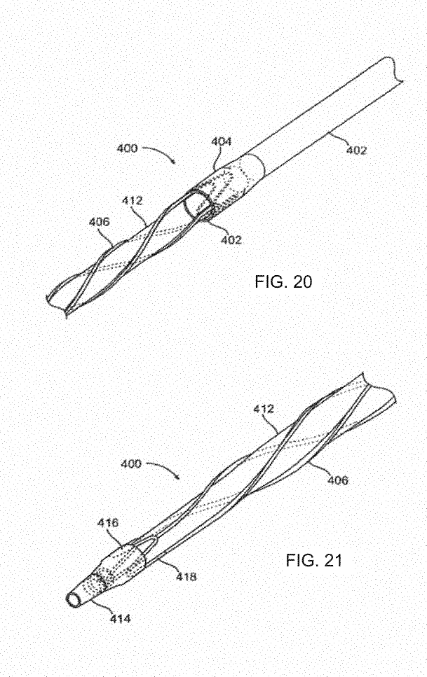

FIGS. 20 and 21 illustrate scoring cage 400 positioned over an expandable dilation balloon 412. As shown in FIG. 21, distal end 418 of the scoring structure may be coupled to the distal tip 414 of the catheter body by an end cap 416. The end cap 416 may be composed of a compatible polymer and thermally bonded with the catheter body to fix distal end 418 of the scoring structure to the catheter body.

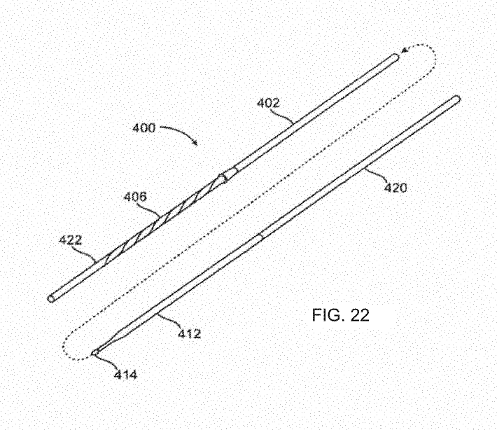

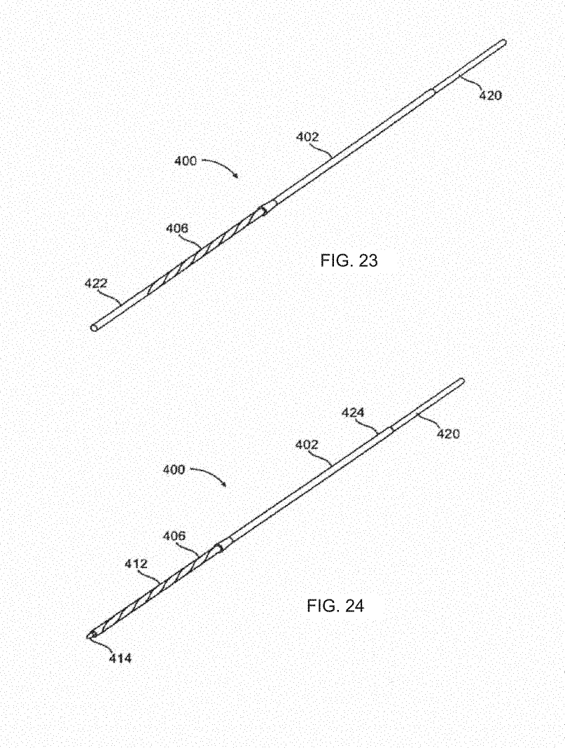

Now referring to FIGS. 22-24, a method is illustrated for mounting an expandable scoring cage 406 over a balloon catheter. The scoring cage 406 is pre-expanded by loading it over an insertion tube 422 that has an inner diameter slightly larger than the outer diameter of the balloon 412. A catheter body 420 having a balloon 412 is then inserted into the inner diameter of the insertion tube 422 and advanced until the balloon 412 is appropriately positioned with respect to the scoring structure 406, as illustrated in FIG. 23. The insertion tube 422 is then pulled back to allow the expanded scoring structure to collapse over the balloon 412 and the catheter body 420, as shown in FIG. 24. The scoring structure 406 may then be secured at its distal end 418 to the distal tip 414 of the catheter body 420 and the proximal end 424 of the scoring structure/attachment structure assembly to a medial location on the catheter body 420.

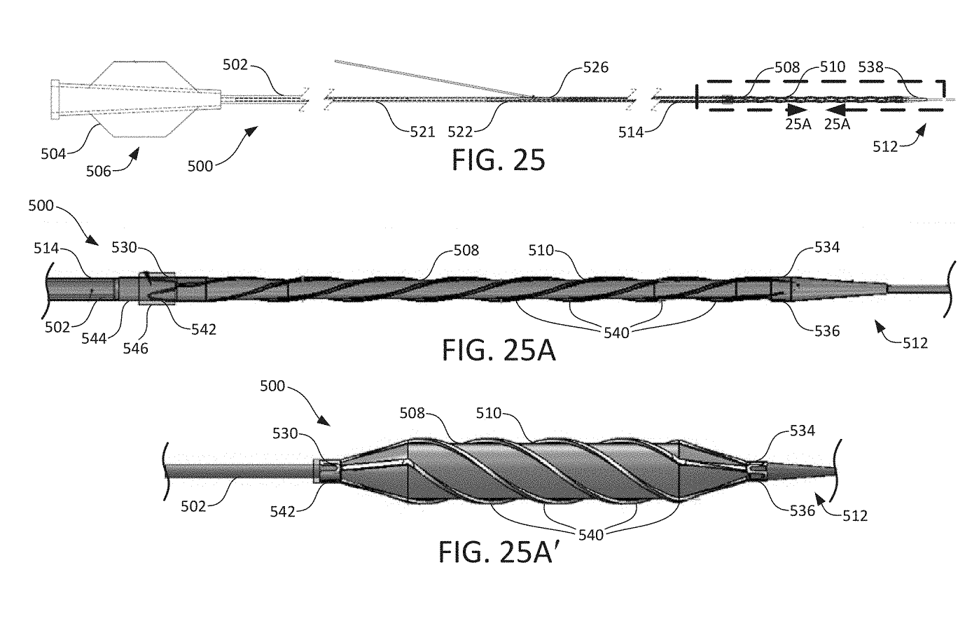

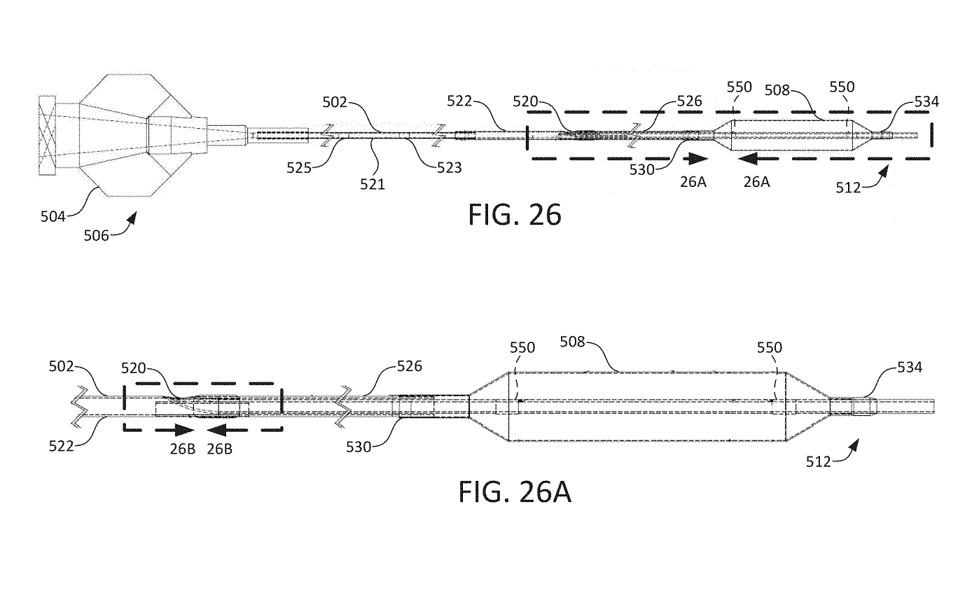

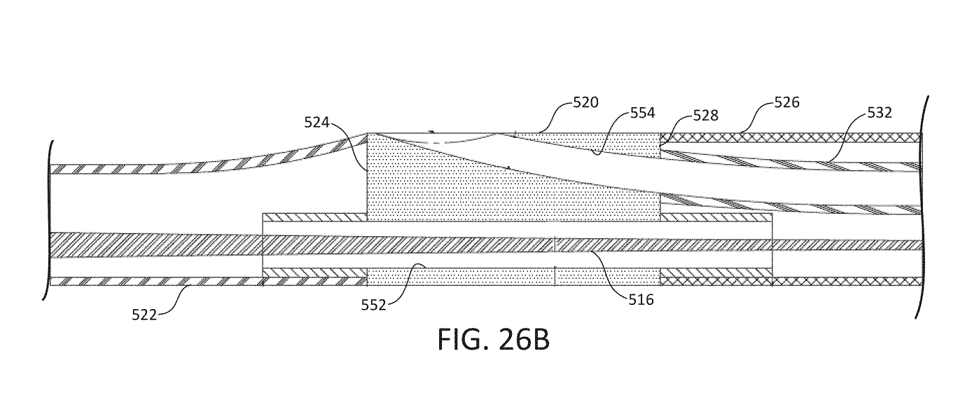

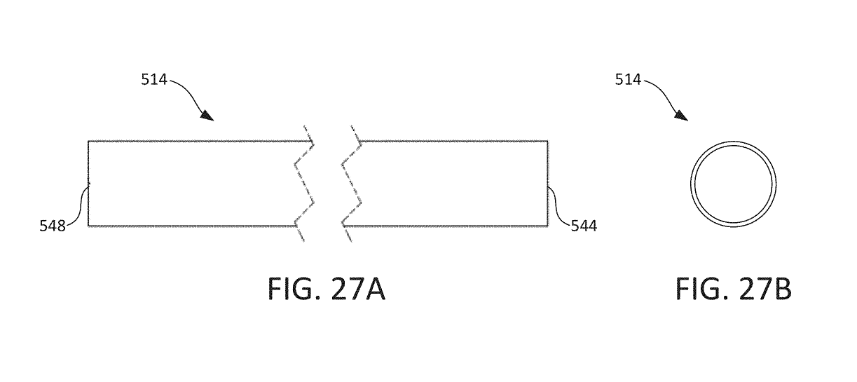

Referring now to FIGS. 25-27B, another embodiment of an angioplasty catheter 500 is illustrated. The angioplasty catheter 500 includes a catheter body 502 that carries a hub 504 near at the proximal end 506 of the catheter 500. The catheter body 502 also carries a expandable shell 608 and an external structure 510 near the distal end 512 of the catheter 500. The catheter body 502 also carries an external axially distensible attachment structure 514 or compliance tube (see FIGS. 26, 27A, and 27B), an internal core wire 516 (see FIGS. 25B and 26B), and a rapid exchange port 520 (see FIG. 26B).

The catheter body 502 includes a flexible proximal sheath 521 (for example, a polytetrafluoroethylene (PTFE)-coated 304 stainless steel hypotube) that extends distally from the hub 504. As shown in FIG. 26, the flexible proximal sheath 521 includes a distal marker 523 and a proximal marker 525. The markers 523 and 525 are defined by uncoated sections of the proximal sheath 521. The markers 523 and 525 act as indicators for the position of the expandable shell 608 relative to a guide catheter (not shown) used to deliver the balloon 508 to a treatment site within a patient. Specifically, the distal marker 523 passes by the surgeon's hand and/or enters the guide catheter when the distal end 512 of the angioplasty catheter 500 is flush with the distal end of the guide catheter, and the proximal marker 525 passes by the surgeon's hand and/or enters the guide catheter when the expandable shell 608 has exited the guide catheter (that is, the expandable shell 608 is distal to the guide catheter).

Opposite the hub 504, the flexible proximal sheath 521 couples to a flexible intermediate sheath 522, which extends to the proximal end 524 of the rapid exchange port 520 (see FIG. 26B). The catheter body 502 also includes a flexible outer sheath 526 that extends from the distal end 528 of the rapid exchange port 520 to the proximal end 530 of the expandable shell 608. The catheter body 502 further includes a flexible inner sheath 532 (see FIG. 26B) that extends from the distal end 528 of the rapid exchange port 520, through the expandable shell 608, and to the distal end 534 of the expandable shell 608.

The external structure 510 is held over the expandable shell 608 and is fixed at the distal end 536 to the catheter body 502 via an adhesively and/or thermally bonded and distally tapering cap 538. The distal end 536 of the external structure 510 couples to four helically extending scoring elements 540, which in turn couple to the proximal end 542 of the external structure 510. The proximal end 542 of the external structure 510 is connected to the distal end 544 of the attachment structure 514 via an adhesively and/or thermally bonded collar 546.

The attachment structure 514 may be configured to reduce forces applied on the external structure 510 and the catheter body 502 during expansion and contraction of the expandable shell 608. The attachment structure 514 includes a cylindrical over-tube made of an elastic material. The attachment structure 514 generally has an inner diameter that is slightly greater than the outer diameter of the sheath of the catheter body 502. The distal end 544 of the attachment structure 514 is attached to the external structure 510 via the collar 546. The distal end 544 of the attachment structure 514 is free to slide axially and rotationally with respect to the catheter body 502. The proximal end 548 of the attachment structure 514 may be fixed to the catheter body 502 via an adhesive and/or thermal bond.

As the expandable shell 608 expands, the external structure 510 expands in circumference and contracts axially along the catheter body 502, applying a tensile force to the attachment structure 514. The attachment structure 514, being fixed to the proximal end 542 of the external structure 510, axially stretches to accommodate the axial movement of the external structure 510. The external structure 510 also tends to rotate about the catheter body 502, causing a torsional force. The distal end 544 of the attachment structure 514 rotates through the full range of motion of the external structure 510 to accommodate the torsional force, while the proximal end 548 remains stationary with respect to catheter body 502.

The attachment structure 514 has a length of about 61.5 mm, an external diameter of 1.1 mm, and an internal diameter of 1.0 mm. The attachment structure 514 comprises Pebax™.

The catheter body 502 carries two radiopaque markers 550 (see FIGS. 26 and 26A) within the expandable shell 608 to facilitate locating the position of the expandable shell 608 under fluoroscopy. The radiopaque markers 550 are positioned near the proximal end 530 and the distal end 534 of the expandable shell 608.

The catheter body 502 carries the core wire 516 (see FIGS. 25B and 26B) therein. The core wire 516 extends from the distal end of the proximal sheath 521 to the distal end 512 of the catheter 500 to facilitate advancing the catheter 500 in the vasculature of the subject. The core wire 516 has a diameter that tapers inwardly proceeding from the proximal end 506 to the distal end 512 of the catheter 500.

The rapid exchange port 520 (see FIG. 26B) is disposed between the intermediate sheath 522 and the outer sheath 526 of the catheter body 502. The core wire 516 extends through an internal lumen 552 of the rapid exchange port 520. The rapid exchange port 520 also includes a guidewire lumen 554 having a transverse opening to facilitate receiving a guidewire 556. The guidewire lumen 554 delivers the guidewire 556 to the inner sheath 532.

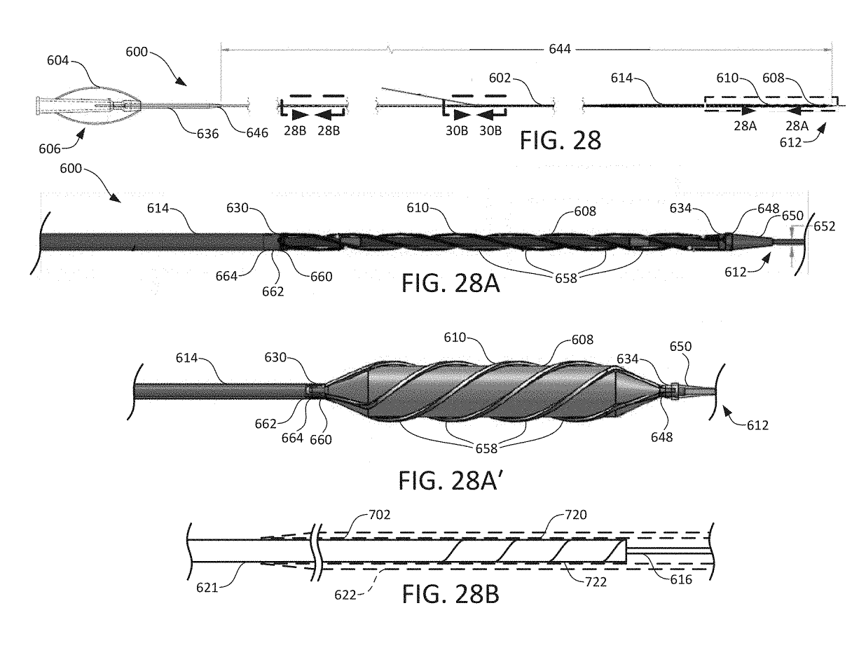

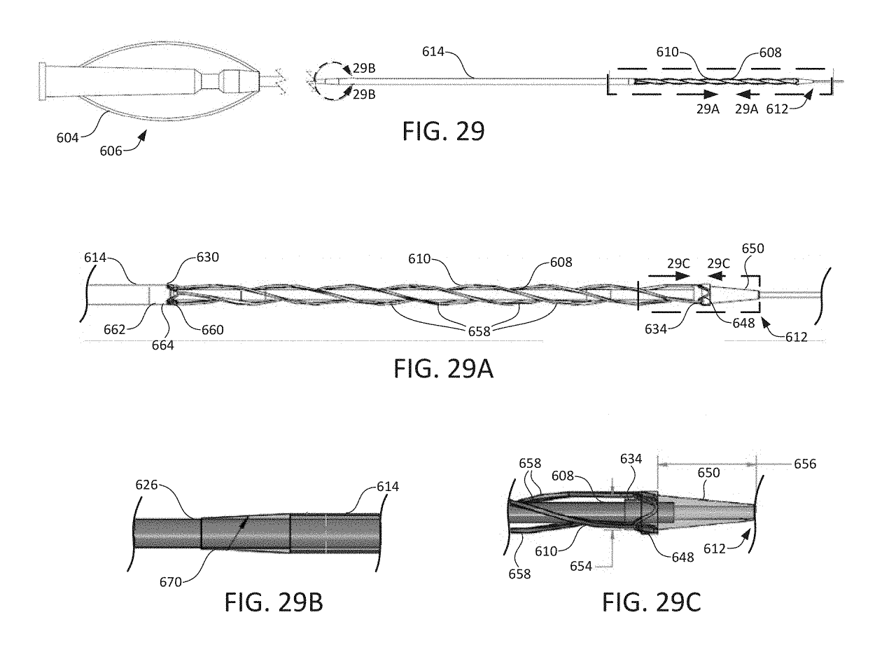

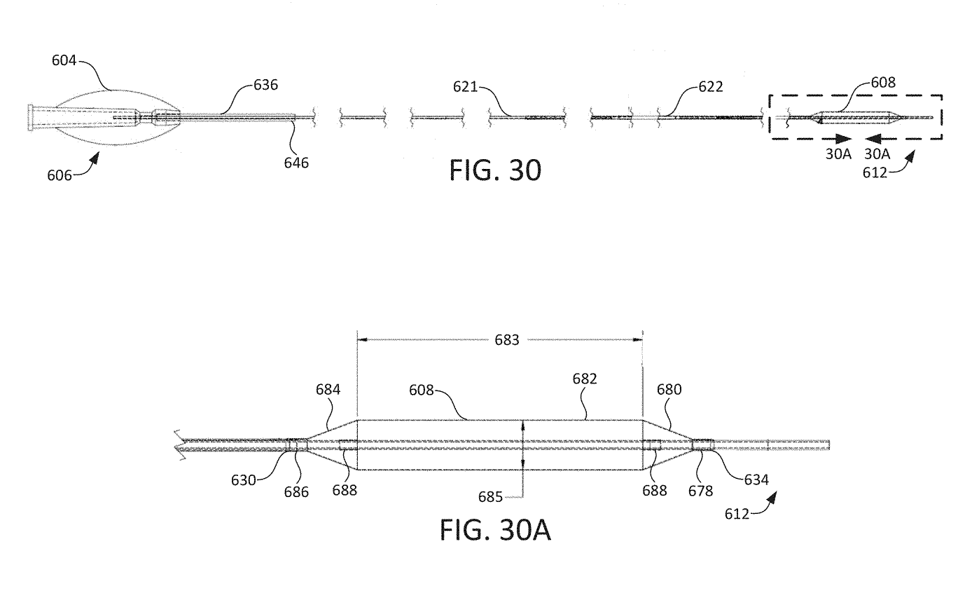

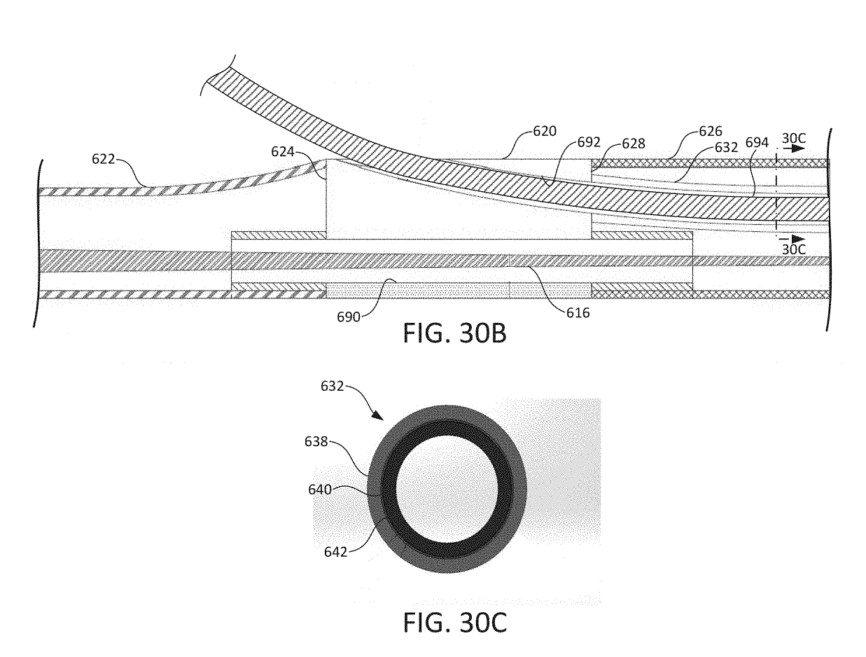

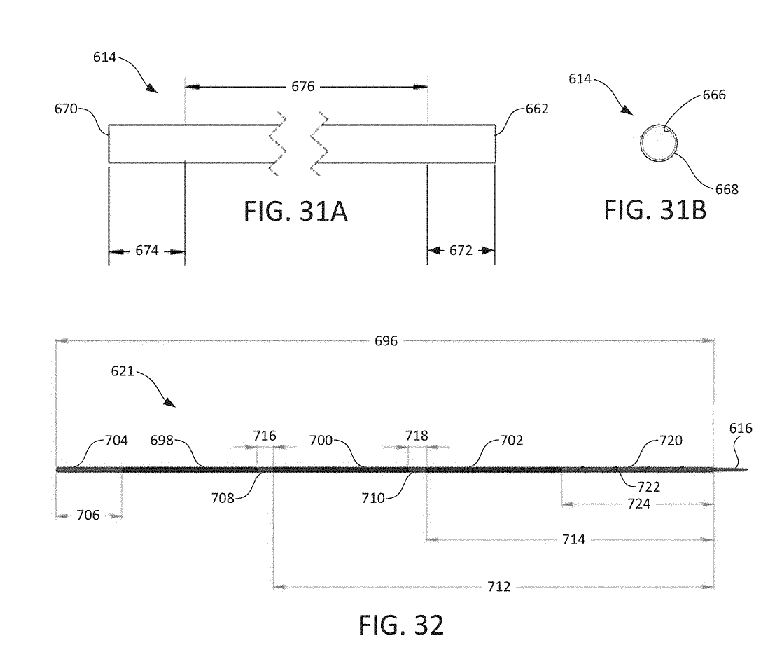

Referring now to FIGS. 28-32, another embodiment of an angioplasty catheter 600 is illustrated. The angioplasty catheter 600 includes a catheter body 602 that carries a hub 604 near at the proximal end 606 of the catheter 600. The catheter body 602 also carries an expandable shell 608 (for example, a dilatation balloon) and an external structure 610 near the distal end 612 of the catheter 600. The catheter body 602 also carries an external axially distensible attachment structure 614 or compliance tube (see FIGS. 29A, 29B, 31A, and 31B), an internal core wire 616 (see FIGS. 28B and 30B), and a rapid exchange port 620 (see FIG. 30B).

The catheter body 602 includes a flexible proximal sheath 621 that extends distally from the hub 604. Opposite the hub 604, the flexible proximal sheath 621 couples to a flexible intermediate sheath 622 that extends from the hub 604 to the proximal end 624 of the rapid exchange port 620 (see FIGS. 28, 28B, and 30B). The catheter body 602 also includes a flexible outer sheath 626 that extends from the distal end 628 of the rapid exchange port 620 to the proximal end 630 of the expandable shell 608. The catheter body 602 further includes a flexible inner sheath 632 (see FIGS. 30B and 30C) that extends from the distal end 628 of the rapid exchange port 620, through the expandable shell 608, and to the distal end 634 of the expandable shell 608. The intermediate sheath 622 also carries a strain relief 636 (see FIG. 28) that extends from the hub 604 toward the distal end 612 of the catheter 600. The catheter 600 may have a working length 644 of about 137 cm (that is, 137 cm±3 cm) between a distal end 646 of the strain relief 636 and the distal end 612 of the catheter 600. The strain relief 636 may have a length of about 30 mm (that is, 30 mm±3 mm).

In some embodiments and referring specifically to FIG. 30C, the inner sheath 632 includes an inner layer 638, a tie layer 640 surrounding the inner layer 638, and an outer layer 642 surrounding the tile layer 640. In some embodiments, the inner layer 638 may comprise high-density polyethylene, the tie layer 640 may comprise a maleic anhydride modified linear low-density polyethylene adhesive resin (for example, OREVAC® 18300 available from Arkema Functional Polyolefins), and the outer layer 642 may comprise a polymer (for example, Pebax™ 63D) to facilitate flexibility and trackability.

The external structure 610 is held over the expandable shell 608 and is fixed at the distal end 648 to the catheter body 602 via an adhesively and/or thermally bonded and distally tapering cap 650. In some embodiments and referring to FIGS. 28A and 29C, the cap 650 has a distal end diameter 652 (and provides the catheter 600 with an entry profile) that is less than about 0.021″ (that is, 0.021″±0.003″), a proximal end diameter 654 that is less than about 0.038″ (that is, 0.038″±0.003″), and a tapering length 656 of about 3.25 mm (that is, 3.25 mm±0.5 mm). In some embodiments, the cap 650 comprises a polymer (for example, Pebax™) that carries a lubricant (for example, barium sulfate) to facilitate movement of the catheter within the vasculature of a subject. As a specific example, the cap 650 may comprise Pebax™ 5533 with 20 percent barium sulfate.

The distal end 648 of the external structure 610 includes a serpentine ring that has a length, for example, of about 0.75 mm (that is, 0.75 mm±0.05 mm). Proximal to the distal end 648 of the external structure 610, the external structure 610 includes a plurality of helically extending scoring elements 658. Illustratively, the external structure 610 includes four helically extending scoring elements 658. The proximal end 660 of the external structure 610 includes a serpentine ring that has a length, for example, of about 0.75 mm (that is, 0.75 mm±0.05 mm). The proximal end 660 of the external structure 610 is connected to the distal end 662 of the attachment structure 614 via an adhesively and/or thermally bonded collar 664.

The attachment structure 614 may be configured to reduce forces applied on the external structure 610 and the catheter body 602 during expansion and contraction of the expandable shell 608. In some embodiments, the attachment structure 614 includes a cylindrical over-tube made of an elastic material (for example, PebaxT™ 6333 SA 01). The attachment structure 614 generally has an inner diameter 666 that is slightly greater than the outer diameter 668 of the sheath of the catheter body 602. Referring to FIGS. 31A and 31B, the attachment structure 614 may have an inner diameter 666 of about 0.040″ (that is, 0.040″±0.005″) and outer diameter 668 of about 0.044″ (that is, 0.044″±0.005″). The distal end 662 of the attachment structure 614 is attached to the external structure 610 via the collar 664. The distal end 662 of the attachment structure 614 is free to slide axially and rotationally with respect to the catheter body 602. The proximal end 670 of the attachment structure 614 (see FIG. 29B) may be fixed to the catheter body 602 via an adhesive and/or thermal bond.

In some embodiments, the attachment structure 614 includes a hydrophilic coating to facilitate pushability and trackability of the catheter 600. The hydrophilic coating may be, for example, HydroSleek 2 (BaseCoat: 50-F000-0082(B23KX̂2); TopCoat: 10-F000-0138, Hydak T-070) available from Biocoat Incorporated of Horsham, Pa. In some embodiments, the attachment structure 614 includes uncoated portions 672 and 674 near the distal end 662 and the proximal end 670, respectively, and a coated portion 676 disposed therebetween. In some embodiments, the uncoated portions 672 and 674 have lengths of about 7.5 mm (that is, 7.5 mm±5.0 mm) and the coated portion 676 has a length of about 68 mm (that is, 68 mm±3 mm).

Referring specifically to FIG. 30A, the expandable shell 608 includes a distal leg 678 that is coupled to the distal end 634 of the expandable shell 608. The distal leg 678 may have a length of about 1.5 mm (that is, 1.5 mm±0.5 mm). The distal leg 678 is coupled to a distal tapering portion 680 of the expandable shell 608, which in turn couples to a working portion 682 of the expandable shell 608. The working portion 682 may have various combinations of length 683 and diameter 685. For example, the length 683 and diameter 685 may be as shown in Table 1.

TABLE 1
Exemplary combinations of working
portion length 683 and diameter 685.

| Working Portion | Working Portion |
|---|---|
| Length 683 | Diameter 685 |
| 6 ± 0.5 mm | 2.1 + 0.1/−0.2 mm |
| 10 ± 0.5 mm | 2.1 + 0.1/−0.2 mm |
| 15 ± 0.5 mm | 2.1 + 0.1/−0.2 mm |
| 20 ± 0.5 mm | 2.1 + 0.1/−0.2 mm |
| 6 ± 0.5 mm | 2.6 + 0.1/−0.2 mm |
| 10 ± 0.5 mm | 2.6 + 0.1/−0.2 mm |
| 15 ± 0.5 mm | 2.6 + 0.1/−0.2 mm |
| 20 ± 0.5 mm | 2.6 + 0.1/−0.2 mm |
| 6 ± 0.5 mm | 3.1 + 0.1/−0.2 mm |
| 10 ± 0.5 mm | 3.1 + 0.1/−0.2 mm |
| 15 ± 0.5 mm | 3.1 + 0.1/−0.2 mm |
| 20 ± 0.5 mm | 3.1 + 0.1/−0.2 mm |
| 6 ± 0.5 mm | 3.6 + 0.1/−0.2 mm |
| 10 ± 0.5 mm | 3.6 + 0.1/−0.2 mm |
| 15 ± 0.5 mm | 3.6 + 0.1/−0.2 mm |
| 20 ± 0.5 mm | 3.6 + 0.1/−0.2 mm |

The working portion 682 couples to a proximal tapering portion 684 opposite the distal tapering portion 680. The proximal tapering portion 684 couples to a proximal leg 686, which is in turn coupled to the distal end 662 of the attachment structure 614. The proximal leg 686 may have a length of about 1.5 mm (that is, 1.5 mm±0.5 mm).

As the expandable shell 608 expands, the external structure 610 expands in circumference and contracts axially along the catheter body 602, applying a tensile force to the attachment structure 614. The attachment structure 614, being fixed to the proximal end 660 of the external structure 610, axially stretches to accommodate the axial movement of the external structure 610. The external structure 610 also tends to rotate about the catheter body 602, causing a torsional force. The distal end 662 of the attachment structure 614 rotates through the full range of motion of the external structure 610 to accommodate the torsional force, while the proximal end 670 remains stationary with respect to catheter body 602.

The catheter body 602 carries two radiopaque markers 688 (see FIG. 30A) within the expandable shell 608 to facilitate locating the position of the expandable shell 608 under fluoroscopy. The radiopaque markers 688 are positioned in the distal tapering portion 680 and the proximal tapering portion 684.

The catheter body 602 carries the core wire 616 (see FIGS. 28B and 30B) therein. The core wire 616 extends from the distal end of the proximal sheath 621 to the distal end 612 of the catheter 600 to facilitate advancing the catheter 600 in the vasculature of the subject. The core wire 616 has a diameter that tapers inwardly proceeding from the proximal end 606 to the distal end 612 of the catheter 600.

The rapid exchange port 620 (see FIG. 30B) is disposed between the intermediate sheath 622 and the outer sheath 626 of the catheter body 602. The core wire 616 extends through an internal lumen 690 of the rapid exchange port 620. The rapid exchange port 620 also includes a guidewire lumen 692 having a transverse opening to facilitate receiving a guidewire 694. The guidewire lumen 692 delivers the guidewire 694 to the inner sheath 632.

The proximal sheath 621 is shown separately in FIG. 32. In some embodiments, the proximal sheath 621 extends distally from the strain relief 636 by about 41.5 in. (that is, 41.5 in.±0.5 in.). In some embodiments, the proximal sheath 621 may be, for example, a hypotube comprising stainless steel, nitinol, or the like. As a specific example, the proximal sheath 621 may be 304 stainless steel. The proximal sheath 621 may have a length 696 of about 1223 mm (that is, 1223 mm±5 mm). The proximal sheath 621 may include one or more coated sections (for example, three PTFE-coated (specifically, Teflon-coated) sections 698, 700, and 702) to provide lubricity.

The proximal sheath 621 may also include one or more uncoated sections. Specifically, the proximal sheath 621 includes a proximal uncoated section 704 to which the strain relief 636 and/or the hub 604 is coupled (for example, via overmolding). The proximal uncoated section 704 may have a length 706 of about 30 mm (that is, 30 mm±5 mm). The proximal sheath 621 also includes a first intermediate uncoated section 708 between the first and second coated sections 698 and 700 and a second intermediate uncoated section 710 between the second and third coated sections 700 and 702. The intermediate uncoated sections 708 and 710 act as indicators for the position of the expandable shell 608 relative to a guide catheter (not shown) used to deliver the shell 608 to a treatment site within a patient. Specifically, the second intermediate uncoated section 710 passes by the surgeon's hand and/or enters the guide catheter when the distal end 612 of the angioplasty catheter 600 is flush with the distal end of the guide catheter, and the first intermediate uncoated section 708 passes by the surgeon's hand and/or enters the guide catheter when the expandable shell 608 has exited the guide catheter (that is, the expandable shell 608 is distal to the guide catheter). The first intermediate uncoated section 708 may be offset from the distal end of the proximal sheath 621 by about 740 mm (that is, 740 mm±10 mm). The second intermediate uncoated section 710 may be offset from the distal end of the proximal sheath 621 by about 640 mm (that is, 640 mm±10 mm). The first intermediate uncoated section 708 and the second intermediate uncoated section 710 may have lengths 716 and 718, respectively, of about 5 mm (that is, 5 mm±0.5 mm).

The proximal sheath 621 also includes a distal uncoated section 720. The distal uncoated section 720 may have a first bending stiffness, and the remainder of the proximal sheath 621 may have a second bending stiffness that is greater than the first bending stiffness. The relative bending flexibility of the distal uncoated section 720 may be provided, for example, by a spiral cut 722 formed therein. The spiral cut 722 may have a variety of dimensions. As a specific example, the spiral cut 722 may have a kerf width of about 0.25 mm (that is, 0.25 mm±0.2 mm), a pitch of about 3 mm (that is, 3 mm±0.5 mm), and a total length of about 75 mm (that is, 75 mm±2 mm). The distal uncoated section 720 may have a length 724 of about 100 mm (that is, 100 mm±10 mm).

Any of the devices described herein may include one or more of the stabilizing struts described and shown in U.S. patent application Ser. No. 14/048,955, the full disclosure of which is incorporated herein by reference. The full disclosure of U.S. patent application Ser. No. 14/275,264 is also incorporated herein by reference.

It will be appreciated by persons skilled in the art that the present invention is not limited to what has been particularly shown and described hereinabove. Alternate embodiments are contemplated that fall within the scope of the invention.

What is claimed is:

1. An angioplasty catheter comprising:
a balloon;
a helical scoring structure having a distal end and a proximal end carried over the balloon, wherein the helical scoring structure comprises a plurality of helical elements that longitudinally extend from the distal end to the proximal end, wherein the plurality of helical elements are coupled together at the distal end and the proximal end; and
a tip coupled to the distal end of the helical scoring structure, the tip tapering inwardly proceeding distally, and the tip comprising a polymer carrying a lubricant.

2. The angioplasty catheter of claim 1, wherein the lubricant comprises barium sulfate.

3. The angioplasty catheter of claim 1, wherein the tip has a tapering length of about 3.25 mm.

4. The angioplasty catheter of claim 1, wherein the angioplasty catheter further comprises a sheath coupled to the balloon and the helical scoring structure.

5. The angioplasty catheter of claim 4, wherein the sheath comprises a hypotube.

6. The angioplasty catheter of claim 5, wherein the hypotube comprises a spiral cut.

7. The angioplasty catheter of claim 6, wherein the spiral cut comprises a kerf width of about 0.25 mm, a pitch of about 3 mm, and a total length of about 75 mm.

8. An angioplasty catheter comprising:
a sheath comprising a hypotube having a spiral cut;
a balloon coupled to the sheath; and
a helical scoring structure having a distal end and a proximal end carried over the balloon, wherein the helical scoring structure comprises a plurality of helical elements that longitudinally extend from the distal end to the proximal end, wherein the plurality of helical elements are coupled together at the distal end and the proximal end.

9. The angioplasty catheter of claim 8, wherein the spiral cut comprises a kerf width of about 0.25 mm, a pitch of about 3 mm, and a total length of about 75 mm.

10. The angioplasty catheter of claim 8, further comprising a tip coupled to the distal end of the helical scoring structure, the tip tapering inwardly proceeding distally, and the tip comprising a polymer carrying a lubricant, wherein the lubricant comprises barium sulfate.

11. A method of dilating a stenosed region in a blood vessel, the method comprising the steps of:
introducing an angioplasty catheter into the blood vessel, the angioplasty catheter comprising:
a balloon;
a helical scoring structure having a distal end and a proximal end carried over the balloon, wherein the helical scoring structure comprises a plurality of helical elements that longitudinally extend from the distal end to the proximal end, wherein the plurality of helical elements are coupled together at the distal end and the proximal end;
a tip coupled to the distal end of the helical scoring structure, the tip tapering inwardly proceeding distally, and the tip comprising a polymer carrying a lubricant;
expanding the balloon to dilate the helical scoring structure within the stenosed region within the blood vessel, wherein the proximal end of the helical scoring structure moves distally and the helical scoring structure shortens to accommodate such distal movement of the proximal and of the helical scoring structure as the balloon is expanded;
holding the expanded helical scoring structure in place to disrupt the stenosis; and deflating the balloon causing the helical scoring structure to collapse.

12. The method of claim 11, wherein the lubricant comprises barium sulfate.

13. The method of claim 11, wherein the tip has a tapering length of about 3.25 mm.

14. The method of claim 11, wherein the angioplasty catheter further comprises a sheath coupled to the balloon and the helical scoring structure.

15. The method of claim 14, wherein the sheath comprises a hypotube.

16. The method of claim 15, wherein the hypotube comprises a spiral cut.

17. The method of claim 16, wherein the spiral cut comprises a kerf width of about 0.25 mm, a pitch of about 3 mm, and a total length of about 75 mm.

18. The method of claim 11, wherein the helical scoring structure accommodates rotation of the plurality of helical elements as the balloon is expanded.
